(19) United States
(12) Patent Application Publication
(10) Pub. No.: US 2025/0325166 A1
(43) Pub. Date: Oct. 23, 2025

(54) MEDICAL IMAGING DEVICE, METHOD FOR OPERATING SAME, AND METHOD OF MEDICAL IMAGING

(71) Applicant: KARL STORZ SE & Co. KG, Tuttlingen (DE)

(72) Inventors: Lukas BUSCHLE, Tuttlingen (DE); Werner GÖBEL, Tuttlingen (DE)

(21) Appl. No.: 18/872,062

(22) PCT Filed: Jun. 5, 2023
(86) PCT No.: PCT/EP2023/065013

§ 371 Date: Dec. 5, 2024

(30) Foreign Application Priority Data
Jun. 10, 2022 (DE) 10 2022 114 606.5

(51) Int. Cl. A61B 1/00

(52) U.S. Cl. CPC A61B 1/00009; A61B 1/00057

(57) ABSTRACT

The invention relates to a medical imaging device (10), comprising: a spatially and spectrally resolving image acquisition unit (12), which comprises at least one optical system (14) and at least one image acquisition sensor system (16) coupled to the optical system, which are designed to perform an image acquisition of an image region (18), in the course of which spatially and spectrally resolved image data are generated which comprise both spatial and spectral information; an evaluation unit (20), which is designed to perform an analysis of the spatially and spectrally resolved image data that is based on spatial and spectral information and based on at least one analysis parameter calculated from the spatially and spectrally resolved image data; a fault detection unit (22), which is designed to detect the presence of a fault in the course of the image acquisition, independently of the analysis of the image data and the calculated analysis parameter, and to determine a fault state of the image acquisition; and an output unit (24), which is designed to generate, according to the fault state, a user output which is based on the analysis parameter.

The invention also relates to a method for operating a medical imaging device (10) and to a method of medical imaging.

The invention relates to a medical imaging device, a method for operating a medical imaging device, and a method for medical imaging.

Medical imaging devices, such as endoscopic or exoscopic devices that produce multispectral or hyperspectral images, are known from the prior art. Multispectral or hyperspectral images have a spectral dimension in addition to two spatial dimensions, such as a conventional image from a camera. The spectral dimension includes multiple spectral bands (wavelength bands). Multispectral and hyperspectral images differ substantially in the number and width of their spectral bands.

Some imaging devices are known for producing such multispectral or hyperspectral images, especially in the context of medical applications. For example, DE 20 2014 010 558 U1 describes a device for acquiring a hyperspectral image of an examination region of a body. The device includes an input lens for generating an image in an image plane and a slit-shaped aperture in the image plane for masking out a slit-shaped region of the image. The light passing through the aperture is spread out by a dispersive element and recorded by a camera sensor. This allows the camera sensor to record a plurality of spectra, each with an associated spatial coordinate, along the longitudinal direction of the slit-shaped aperture. The device described is further configured to record further spectra along the longitudinal direction of the slit-shaped aperture in a direction different from the longitudinal direction of the slit-shaped aperture. The method underlying this disclosure for generating multispectral or hyperspectral images is also known as the so-called pushbroom method.

In addition to the pushbroom method, there are other methods for generating multispectral or hyperspectral images. In the so-called whiskbroom method, the region under study or else the object is scanned point by point and a spectrum is obtained for each point. In contrast, the staring method involves taking multiple images with the same spatial coordinates. Different spectral filters and/or illumination sources are used from image to image to resolve spectral information. Furthermore, there are methods according to which a two-dimensional multi-color image is broken down into a plurality of individual spectral images using suitable optical elements, such as optical slicers, lenses and prisms, which images are simultaneously acquired on different detectors or detector regions. This is sometimes referred to as the snapshot approach.

As described in DE 10 2020 105 458 A1, multispectral and hyperspectral imaging devices are particularly suitable as endoscopic imaging devices. In this context, multispectral and/or hyperspectral imaging is a fundamental field of application, for example for diagnostics and for assessing the success or quality of an intervention.

The robustness and low susceptibility to errors of the imaging device are of central importance to allow a user to achieve a high quality and reliability of an imaging-device-based interpretation of multispectral and hyperspectral images. The robustness and susceptibility to errors may depend on the influence of faults during the image acquisition process. Known faults in this context can be, for example, smoke after thermal manipulation of tissue, soiled or fogged lenses of the imaging device or inorganic materials in the image region (instruments, clamps, threads, etc.). These faults can lead to image acquisition faults that can complicate reliable interpretation of multispectral and hyperspectral images.

Based on the prior art, the object of the present invention is to provide an imaging device and an imaging method by means of which faults and/or fault states can be detected during image acquisition.

An imaging device according to the invention may comprise a spatially and spectrally resolving image acquisition unit which comprises at least one optical system and at least one image acquisition sensor system coupled to the optical system, which are configured to carry out image acquisition in which spatially and spectrally resolved image data are generated that comprise both spatial and spectral information. Furthermore, the imaging device may comprise an evaluation unit which is configured to create an analysis of the spatially and spectrally resolved image data that is based on spatial and spectral information and based on at least one analysis parameter calculated from the spatially and spectrally resolved image data. In addition, the imaging device may comprise a fault detection unit configured to detect the presence of a fault in the image acquisition and to determine a fault state of the image acquisition. Finally, the imaging device may comprise an output unit configured to generate a user output based on the analysis parameter in accordance with the fault state.

According to one aspect, a medical system is provided that comprises the imaging device and a medical instrument.

Furthermore, the present invention may relate to a method for operating a medical imaging device, wherein the medical imaging device comprises a spatially and spectrally resolving image acquisition unit. The image acquisition unit comprises at least one optical system and at least one image acquisition sensor system coupled to the optical system, which are configured to carry out an image acquisition of an image region in which spatially and spectrally resolved image data are generated.

The image data comprise both spatial and spectral information. This can be an imaging device according to the invention. The method may comprise the step of acquiring spatially and spectrally resolved image data of an image region by means of the medical imaging device. Furthermore, the method may comprise the step of creating an analysis of the spatially and spectrally resolved image data that is based on spatial and spectral information and based on at least one analysis parameter calculated from the spatially and spectrally resolved image data. Furthermore, the method may comprise performing a fault detection in which the presence of a fault in the image acquisition is detected and in which a fault state of the image acquisition is determined. The method may further comprise the step of generating a user output in accordance with the fault state based on the analysis parameter.

Furthermore, the present invention may include a method for medical imaging. The method can be carried out with an imaging device according to the invention and/or with a medical system according to the invention. Such a method may include performing an image acquisition of an image region, thereby generating spatially and spectrally resolved image data comprising both spatial and spectral information.

Furthermore, a step of such a method can be the creation of an analysis of the spatially and spectrally resolved image data that is based on spatial and spectral information and based on at least one analysis parameter calculated from the spatially and spectrally resolved image data. Furthermore, the method may comprise the step of performing a fault detection in which the presence of a fault in the image acquisition is detected and in which a fault state of the image acquisition is determined. The method may further comprise the step of generating a user output in accordance with the fault state based on the analysis parameter.

The features according to the invention allow a reliable implementation and/or assessment of diagnostic and/or therapeutic actions. In particular, a high degree of quality of multispectral and/or hyperspectral imaging can be achieved because any faults in the display of both spatially and spectrally resolved information can be detected in the form of spatially and spectrally resolved image data. By indicating image acquisition faults and image acquisition fault states to the user as such, misinterpretation by the user can be avoided. Referring to an application example in which the present invention is part of a medical system, misinterpretation of physiological tissue parameters—such as, inter alia, oxygen saturation, blood, water or fat content, which form the basis for carrying out and/or assessing therapeutic and/or diagnostic measures—can be prevented.

The fault detection unit may be configured to detect the presence of a fault in the image acquisition and to determine a fault state of the image acquisition independently of the analysis of the image data and the calculated analysis parameter. Alternatively or additionally, in the method according to the invention for operating a medical imaging device and/or in the method according to the invention for medical imaging, the step of carrying out a fault detection may comprise that, during the fault detection, the presence of a fault in the image acquisition is detected and a fault state of the image acquisition is determined independently of the analysis of the image data and the calculated analysis parameter.

The imaging device can be a microscopic, macroscopic and/or exoscopic imaging device. The imaging device can be configured as a microscope, macroscope and/or exoscope and/or comprise such. In some embodiments, the imaging device can be an endoscopic imaging device. The imaging device can be an endoscope device. It can comprise an endoscope and/or an endoscope system and/or be configured as such and/or form at least a part and preferably at least a major part and/or main component of an endoscope and/or an endoscope system. “At least a major part” can mean at least 55%, preferably at least 65%, more preferably at least 75%, particularly preferably at least 85% and most preferably at least 95%, in particular with reference to a volume and/or a mass of an object.

In some embodiments, the imaging device is configured to be insertable into a cavity for inspection and/or observation, for example into an artificial and/or natural cavity, such as into the interior of a body, into a body organ, into tissue or the like. The imaging device can also be configured to be insertable into a housing, casing, shaft, tube or other, in particular artificial, structure for inspection and/or observation.

In particular if the imaging device is an exoscopic imaging device, it may be configured to acquire tissue parameters, images of wounds, images of body parts, etc. For example, the imaging device may be configured to image a surgical field.

The imaging device and in particular the optical system and/or the image acquisition sensor system may be configured for multispectral and/or hyperspectral imaging, in particular for acquiring and/or generating multispectral and/or hyperspectral image data. Multispectral imaging or multispectral image data can refer in particular to such imaging in which at least two, in particular at least three, and in some cases at least five spectral bands can be acquired and/or are acquired independently of one another. Hyperspectral imaging or hyperspectral image data can refer in particular to imaging in which at least 20, at least 50 or even at least 100 spectral bands can be acquired and/or are acquired independently of one another. The imaging device may operate according to the pushbroom method, and/or the whiskbroom method, and/or according to the staring method, and/or according to a snapshot principle.

In some embodiments, the imaging device comprises a white light camera and/or sensor system for white light imaging. The imaging device can be configured for white light imaging in addition to spectrally resolved imaging. A separate optical system and/or a common optical system can be used for this purpose. White light imaging and spectrally resolved imaging can be performed simultaneously or alternately, or sometimes simultaneously and sometimes sequentially.

For some applications it can be advantageous to be able to use a high spectral resolution. Hyperspectral imaging is then recommended. This can be combined with white light imaging. This makes real-time observation possible via a white light image, even if the acquisition of spectrally resolved image data only occurs substantially in real time, i.e., for example, several seconds are needed to create a spectrally resolved image. For some applications it can be advantageous to generate spectral image data in real time. This includes, for example, the generation of a spectrally resolved image in less than a second or even multiple times per second. It can be useful to use multispectral imaging in this case. An optionally lower spectral resolution is then offset by a higher refresh rate. Depending on the application, it can be sufficient to consider only a few different spectral ranges and/or wavelengths, for example two or three or four or generally less than ten. In this case, additional white light imaging can optionally be omitted. Spectrally resolved image data that are acquired in real time or deliver several images per second can also be used for monitoring purposes, wherein it is not absolutely necessary to create a reproducible image for a user, but rather the image data can also be processed in the background.

The medical imaging device can have at least a proximal portion, a distal portion and/or an intermediate portion. The distal portion is in particular configured to be introduced into and/or located in a cavity to be examined in an operating state, for example during the diagnostic and/or therapeutic activity. The proximal portion is in particular configured to be arranged outside the cavity to be examined in an operating state, for example during the diagnostic and/or therapeutic activity. “Distal” should be understood to mean, in particular, facing a patient and/or facing away from a user during use. “Proximal” should be understood to mean, in particular, facing away from a patient and/or facing a user during use. In particular, proximal is the opposite of distal. The medical imaging device in particular has at least one, preferably flexible, shaft. The shaft can be an elongated object. Furthermore, the shaft can at least partially and preferably, at least to a large extent, form the distal portion. An “elongated object” is to be understood in particular as an object whose main extension is at least a factor of five, preferably at least a factor of ten and particularly preferably at least a factor of twenty larger than a largest extension of the object perpendicular to its main extension, i.e. in particular a diameter of the object. A “main extension” of an object should be understood in particular as its longest extension along its main extension direction. A “main extension direction” of a component is to be understood in particular as a direction which runs parallel to a longest edge of a smallest imaginary cuboid which only just completely encloses the component.

The image acquisition unit can be arranged at least partially and preferably at least to a large extent in the region of the proximal portion and/or can form it. In other embodiments, the image acquisition unit may be arranged at least partially and preferably at least to a large extent in the distal portion and/or can form it. Furthermore, the image acquisition unit can be arranged at least partially distributed over the proximal portion and the distal portion. The image acquisition sensor system comprises in particular at least one image sensor. Furthermore, the image acquisition sensor system can also have at least two and preferably several image sensors, which can be arranged one behind the other. Furthermore, the two and preferably several image acquisition sensors can have spectral acquisition sensitivities different from one another so that, for example, a first sensor in a red spectral range, a second sensor in a blue spectral range and a third sensor in a green spectral range is particularly sensitive or comparatively more sensitive than the other sensors. The image sensor can be configured as a CCD sensor and/or a CMOS sensor.

The optical system of the image acquisition unit can comprise suitable optical elements, such as lenses, mirrors, gratings, prisms, optical fibers, etc. The optical system may be configured to guide object light coming from the image region to the image acquisition sensor system, for example to focus and/or project it. The object light can in particular come from illumination of the image region.

The image acquisition unit is in particular configured to generate at least two-dimensional spatial image data. The image acquisition unit can be spatially resolving in such a way that it provides a resolution of at least 100 pixels, preferably of at least 200 pixels, preferably of at least 300 pixels and advantageously of at least 400 pixels in at least two different spatial directions. The image data are preferably at least three-dimensional, wherein at least two dimensions are spatial dimensions and/or wherein at least one dimension is a spectral dimension. From the image data, multiple spatially resolved images of the image region can be obtained, each of which is assigned to different spectral bands. The spatial and spectral information of the image data can be such that an associated spectrum can be obtained for a plurality of spatial pixels.

In some embodiments, the image acquisition unit is configured to generate continuously updated image data. The image acquisition unit can, for example, be configured to generate the image data substantially in real time, which can comprise, for example, generating updated image data at least every 30 seconds, in some cases at least every 20 seconds, and in some cases even at least every 10 seconds or at least every 5 seconds.

The image region may comprise at least a part and/or portion of an imaged object. The image region may concern tissues and/or organs and/or a part of a body of a patient. The image region may relate to a site.

The imaging device may comprise a lighting device that comprises at least one illuminant which is configured to light up and/or illuminate the image region in at least one operating state. The illuminant can comprise a white light source, an, in particular tunable, monochrome light source, a laser, a white light laser, at least one light-emitting diode and/or a light-emitting diode array, at least one laser diode and/or a laser diode array or the like. The lighting device can be formed integrally with the image acquisition unit. In particular, the lighting device can use individual or all components of the optical system of the image acquisition unit and/or have a separate lighting optical system. An illumination light beam can be guided and/or guidable at least in portions coaxially with a measuring light beam.

The analysis may be based on processing both spatial and spectral information to determine the analysis parameter. The analysis parameter may be obtained in particular from spectral information assigned to the image region by means of a mathematical rule. In particular, the analysis parameter can assume a continuous value range of 0-100% or a discrete value range of 0, 1, 2, . . . . The mathematical rule can comprise an algebraic calculation rule and/or a machine learning method, e.g. an AI model. The analysis parameter is based in particular on a spectral value for a specific wavelength, and/or a specific wavelength range, and/or a specific spectral band.

In some embodiments, the analysis may include a parameter image. In particular, the analysis parameter can be spatially resolved. For example, the parameter image can display the value of the analysis parameter as a function of an image coordinate. Alternatively or additionally, the analysis can provide a spectrum. The analysis parameter may, for example, be a spectrally resolved intensity value.

It may be provided that the fault is not recognizable and/or detected from spatial and spectral information, and/or that the fault detection unit does not, or at least does not exclusively, rely on spatial and spectral information. Fault detection may be based on spatial and spectral information. The fault detection may detect faults using a mathematical rule. The mathematical rule may comprise an algebraic calculation rule and/or a machine learning method, for example an AI model. The mathematical rule may also include, for example, at least one filter rule, which can specifically modify image data using algorithms. Furthermore, at least one optical filter may be integrated into the imaging device. Moreover, a mathematical rule may be used to calculate from spatial and spectral image data a comparison parameter, which can be compared to other parameters that empirically are based on fault-free imaging.

Faults may include smoke, soiling and/or wear of the imaging device, in particular the optical system of the imaging device, inorganic materials, in particular instruments, nets, tippers, threads, trocars, overexposed and/or underexposed image regions, contrast agents, motion artifacts, in particular motion artifacts caused by a movement of the imaging device relative to the image region, incorrect white balance in white light imaging provided in some embodiments and/or an endoscope tip located in the trocar during image data generation of the imaging device. Another form of interference can occur when taking pictures in/under water, for example in arthroscopy or urology. Suspended particles in the water, such as urine or blood, can obscure the view of the tissue, which can also lead to distorted parameters. This case is in some ways similar to the case of smoke described here and below, but in a different medium.

The imaging device and in particular the fault detection unit can in each case comprise at least one processor and/or an associated memory with program code that implements the described functions and steps, and/or an associated random access memory, and/or associated ports, and/or data interfaces, and/or an electronic circuit in order to implement the functional units mentioned herein and/or to carry out the method steps mentioned herein. One or more processors, memories, main memories, connections, data interfaces and/or circuits can also be assigned to one or more functional units and/or implement one or more method steps.

The output unit may be configured to output a visual output and/or any other output perceptible by a user. For this purpose, the output unit may comprise suitable components, such as one or more lamps, illuminants, loudspeakers, screens or the like. The output unit can comprise a computer and/or processor, and/or memory, and/or random access memory, and/or ports, and/or a data interface for receiving, processing and outputting unprocessed, preprocessed and/or processed output data.

The imaging device can comprise a display unit that is configured to display an image, in particular a moving image, to a user. The display unit can be part of the output unit and/or form it. The displayed image can be based on the image data. The display unit can comprise a screen and/or control electronics. The display unit can comprise a computer and/or processor, and/or memory, and/or random access memory, and/or ports, and/or a data interface for receiving, processing and outputting unprocessed, preprocessed and/or processed image data and/or display data. The output generation unit can be connected to the display unit via an interface. The generated output can be processed and/or output by the display unit.

The output unit may be configured to change a user output in accordance with the fault state of the imaging device. For example, after detecting a fault state, the user can be alerted to a fault state. In particular, a displayed image of the display unit, spatial and spectral information, which was acquired under the influence of a fault state, may intentionally not be displayed and/or identified. Examples include color coding and/or shaping, such as symbols and/or geometric figures. For example, an image line that was acquired under the influence of a fault state of the imaging device can be marked and output as a black, red or otherwise colored image line. A faulty image line can occur, for example, in the case of imaging using the pushbroom method. Furthermore, it can be provided that, depending on a fault state, image data which were acquired under the influence of a fault state are acquired again. In further embodiments, parameters of the imaging device, in particular of the optical system, the image acquisition sensor system, the lighting device and/or of a white balance can be adapted and/or optimized according to a fault state.

The fault detection unit may be configured to detect the presence of a fault based on an assessment of the spatially and spectrally resolved image data. This makes it easy to determine the presence of a fault from available data. In particular, a fault can be detected independently of white light imaging. The fault detection unit may be configured to process spatial and spectral information according to at least one mathematical rule. For example, by applying a mathematical rule, a comparison parameter can be calculated from the image data and compared to parameters in an expected range for image acquisition without interference. This parameter can be calculated in particular from spectral information.

The fault detection unit can also be configured to detect an image exposure state based on the spatially and spectrally resolved image data and to detect the presence of a fault if the exposure state represents underexposure and/or overexposure at least in portions. This can prevent a user from drawing incorrect conclusions based on distorted spectra. Overexposure occurs, for example, when the image acquisition sensor, in particular a CCD sensor and/or a CMOS sensor, is exposed to an illumination that oversaturates the image acquisition sensor. This may mean that an image-acquisition-sensor-specific threshold for detecting exposure has been exceeded. Underexposure may mean exposure of the image acquisition sensor below a threshold value that is necessary to detect exposure, optionally to distinguish between adjacent image data points and/or image data lines.

A fault may in particular concern the presence of inorganic material in the image region. The fault detection unit may be configured to detect a fault due to inorganic material in the image region. In particular, this can help prevent misinterpretations of medical devices. Inorganic material may include, but is not limited to, instruments, trocars, meshes, clippers and/or sutures. Detection of the inorganic material may be based on processing spectral information, in particular spectral information at at least one specific wavelength, for example two or three or four specific wavelengths, and/or in at least one specific wavelength range, for example two or three or four specific wavelength ranges, and comparing the spectral information to a known spectrum and/or to known selected values associated with inorganic material. This can be particularly useful for distinguishing between organic and inorganic material. In principle, a distinction between organic and inorganic material can be made in many ways, in particular in the ways already described.

Furthermore, the medical imaging device may comprise a video acquisition unit comprising a camera. The camera may be configured to generate video image data of the image region. The video acquisition unit may in particular be arranged in addition to the spectrally resolved imaging. The video acquisition unit may use all units of the imaging device, in particular units, such as the optical system, the illumination device and/or output unit, together with the imaging device. In other embodiments, the video acquisition unit may be partially or completely separate. The fault detection unit may be configured to detect the presence of a fault based on an image analysis of the video image data. This allows the presence of faults to be detected depending on the situation and at least substantially in real time. The spatial and spectral information can be used in addition to the image analysis of the video image data for fault detection. According to the invention, it is also possible to detect a fault by simply analyzing the video image data. In particular, it may be advantageous for the video image data to be available substantially in real time and preferably in real time, i.e., with a time delay of a few milliseconds, for example. The image analysis may comprise a mathematical rule, in particular an algebraic calculation rule and/or a machine learning method, e.g., an AI model.

Incorrect or inaccurate acquisitions can be effectively prevented in particular if the fault detection unit of the medical imaging device is configured to determine a range of motion of the camera relative to the image region based on the image analysis of the video image data and to detect the presence of a fault if the determined range of motion exceeds a threshold value. Because multispectral and hyperspectral imaging of a single image may occur over a period of time during which, due to motion, a spatial coordinate of an image data point at the beginning of the imaging no longer matches the spatial coordinate of the same image data point during the imaging period, a spatial coordinate of the image can no longer be assigned to the image region. By analyzing the video image data, a movement of the camera relative to the image region can be determined during imaging. It can be advantageous if the video image data can be acquired at a higher refresh rate than the multispectral and hyperspectral image data. In particular, a threshold value that represents an acceptable spatial displacement of an image data point during imaging may be defined by a user. According to one embodiment, the range of movement of the camera relative to the image region determined based on the image analysis of the video image data can be compared to this threshold value in order to detect a fault in the image acquisition.

Furthermore, the fault detection unit of the medical imaging device may be configured to detect the presence of soiling and/or fogging on at least part of the optical system of the spatially and spectrally resolving image acquisition unit based on the image analysis of the video image data and to detect the presence of a fault based on the detection of soiling and/or fogging. This can prevent distortions due to soiling and/or fogging on at least part of the optical system.

Furthermore, accurate spectral calibration of the imaging device can be of particular importance to achieve optimal image quality. Spectral calibration can be performed, for example, using white balance. According to one embodiment, the video acquisition unit may also be calibrated by means of a white balance.

The fault detection unit may be configured to assess, based on a comparison of the spatially and spectrally resolved image data and the video image data, whether a white balance of the spatially and spectrally resolved image acquisition unit and a white balance of the video acquisition unit are consistent at least within a predetermined tolerance and to detect the presence of a fault if the predetermined tolerance is exceeded. Because it is usually less complex to perform a white balance of the video acquisition unit than a white balance of the spatially and spectrally resolved image acquisition unit, it may be advantageous to first perform a white balance of the video acquisition unit and to use the video image data as a reference value when comparing them to the spatially and spectrally resolved image data.

If a fault is detected, the user may be prompted to perform a white balance of the spatially and spectrally resolving image acquisition unit again. According to a further embodiment, a white balance of the spatially and spectrally resolving image acquisition unit can be carried out automatically in accordance with a fault state.

Furthermore, the fault detection unit of the medical imaging device may be configured to detect the unexpected presence of a medical instrument in the image region based on the image analysis of the video image data and to detect the presence of a fault in accordance with the detection of an unexpected presence of a medical instrument. This can alert a user that distortions in the images may occur due to medical instruments. In particular, the fault detection unit may be configured to compare a detected medical instrument to a medical instrument expected in the image region and to detect the presence of a fault if the detected medical instrument deviates from the expected medical instrument. Furthermore, depending on the detection of the presence of a medical instrument, the partial region of the image region in which the medical instrument is present can be excluded from the parameter representation. This may include excluding said partial region from underlying calculations.

Furthermore, the fault detection unit of the medical imaging device may comprise a distal end which comprises at least parts of the optical system of the image acquisition unit, wherein the fault detection unit is configured to detect that the image region at least partially comprises an interior of a trocar, and wherein the output unit is configured to generate a user output which contains the information that the distal end is at least partially located within the trocar. The imaging device may be in such close proximity to the trocar during imaging that a fault of the spatial and spectral information may occur. In particular, incident light can be reflected by the trocar, causing a fault of the spatial and spectral information. By detecting unwanted positioning partially within the trocar, corresponding distortions of images can be prevented.

According to one embodiment, the medical imaging device may comprise a fluorescence imaging unit configured to acquire the image region by fluorescence imaging and to generate fluorescence imaging data, wherein the fault detection unit is configured to detect the presence of a fault based on an assessment of the fluorescence imaging data. Individual wavelengths of the spectral information may lie within the emission spectrum of the fluorescence imaging unit and may therefore be subject to influence by the fluorescence imaging unit. For example, the measured intensity of these wavelengths may be altered by the fluorescence imaging unit, which may result in a fault in the image acquisition. The evaluation unit could then calculate incorrect analysis parameters due to the fault. In particular, a tissue located in the image region could be incorrectly assigned to another tissue type.

The fault detection unit of the medical imaging device may further be configured to detect the presence of contrast agent in at least a portion of the image region based on the fluorescence imaging data, wherein the output unit may be configured to generate the user output based on the analysis parameter such that portions of the image region in which the fault detection unit detects a presence of contrast agent are omitted. This makes it possible, for example, to analyze parts of the image region that are not subject to fluorescence-related interference. This is advantageous because it allows multimodal imaging to be achieved, with the potential advantages of each mode being able to be specifically selected for a tissue under investigation. For example, contrast agents can be specifically administered to one tissue type and thus made accessible to fluorescence imaging, while the remaining tissue types within the same image region remain accessible to hyperspectral and/or multispectral imaging.

Furthermore, the medical imaging device may comprise a sensor unit having at least one sensor that is configured to measure at least one measured variable that describes a state of the imaging device and/or an environment of the imaging device, and that is configured to generate a sensor signal that represents the measured variable. In this context, the sensor unit can operate independently of the imaging device. The sensor unit can operate simultaneously with the imaging device and generate a sensor signal and/or before or after the acquisition of multispectral and/or hyperspectral information. The fault detection unit may be configured to detect the presence of a fault based on the sensor signal.

As already described, relative movement between the imaging device and the image region can occur. In particular, to detect this relative movement, the sensor may be an acceleration sensor, wherein the sensor signal represents a movement of the imaging device. In this case, the fault detection unit may be configured to determine a range of motion based on the sensor signal and to detect the presence of a fault if the determined range of motion exceeds a threshold value.

A distance that is too small and/or too large between the imaging device and an object in the image region can also cause interference with image acquisition. Especially in this context, the sensor can be a distance sensor. This may be configured to determine a distance between the imaging device and an object in the image region.

A therapeutic action, such as thermal manipulation of tissue, in particular a vessel sealing or coagulation using an electrosurgical device, such as a radiofrequency coagulation instrument, may produce smoke in the image region. Smoke can alter spectral information of the image region prior to acquisition by the imaging device and thus represent a fault. This can lead to analysis parameters being incorrectly calculated by means of the evaluation unit of the imaging device. The fault detection unit of the medical imaging device may be configured to detect the presence of smoke in the image region based on image analysis and to detect the presence of a fault based on the detection of smoke in the image region. The fault detection unit can detect smoke by analyzing video image data from a video acquisition unit. Smoke detection may be achieved alternatively or additionally by analyzing hyperspectral and/or multispectral information.

The fault detection unit of the medical imaging device may be configured to prevent the acquisition of spatially and spectrally resolved image data by means of the spatially and spectrally resolving image acquisition unit in the event of a fault being detected. For example, the fault detection unit may be configured to at least temporarily deactivate the image acquisition unit and/or to at least temporarily prevent image acquisition. Alternatively or additionally, a user output may be provided to alert the user to a fault state. The user can then, for example, correct the fault state and continue with the acquisition of spatially and spectrally resolved image data and/or continue with the acquisition of spatially and spectrally resolved image data despite the fault state.

A user can be specifically informed of faulty regions of an image in particular if the output unit of the medical imaging device is set up to generate a spatially resolved parameter representation of the image region in accordance with the analysis parameter and for the fault detection unit to mark them as faulty parts of the image region. For example, the part of the image region that is subject to interference can be marked with black or differently colored image data points. Furthermore, a border, strikethrough or other highlighting of the affected image region can be displayed.

The output unit of the medical imaging device may further be configured to provide the user with information on how to correct the fault in the event of a fault being detected. For example, the fault detection unit may be configured to detect the type of fault. This may be based on one and/or a combination of the methods described. For example, after a fault is detected, the user may be prompted to remove a medical instrument from the image region, perform a white balance and/or clean a dirty lens.

In principle, any type of fault can be detected, and the user can be prompted and/or instructed to correct the fault.

The present invention is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using numerical words, such as first, second, third object, etc., these are used to name and/or assign objects. Accordingly, for example, a first object and a third object may be included, but not a second object. However, a number and/or sequence of objects could also be derived using numerical words.

In the drawings:

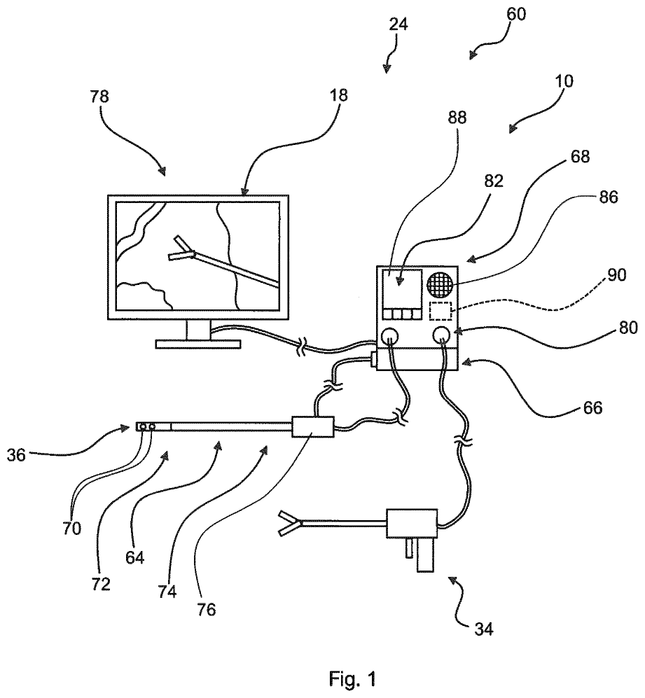

FIG. 1 is a schematic representation of a medical system with a medical imaging device;

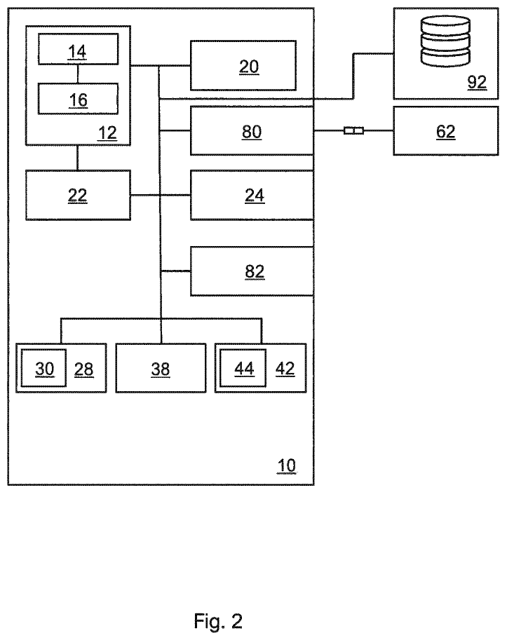

FIG. 2 is a schematic structural diagram of the imaging device;

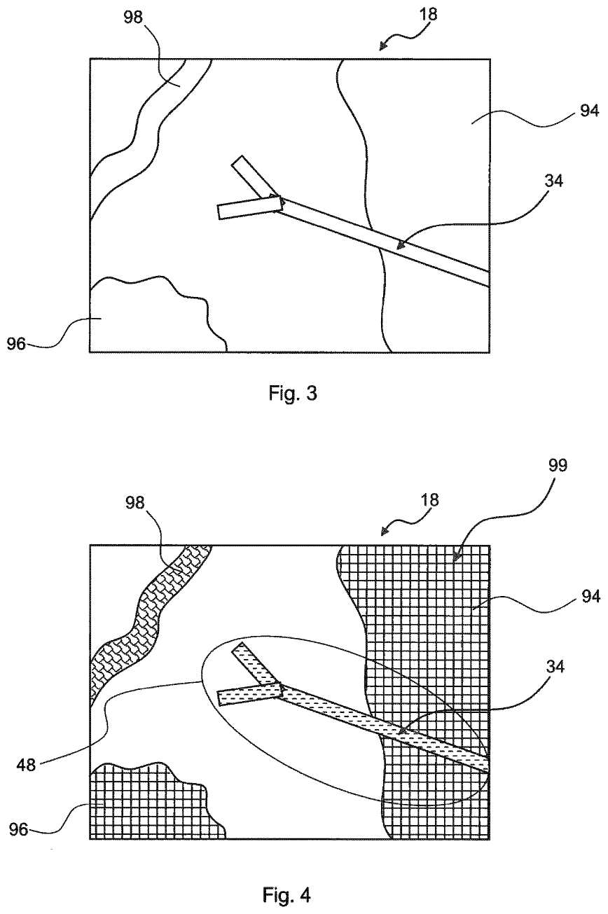

FIG. 3 is a schematic representation of an image region;

FIG. 4 is a further schematic representation of the image region to illustrate a possible fault;

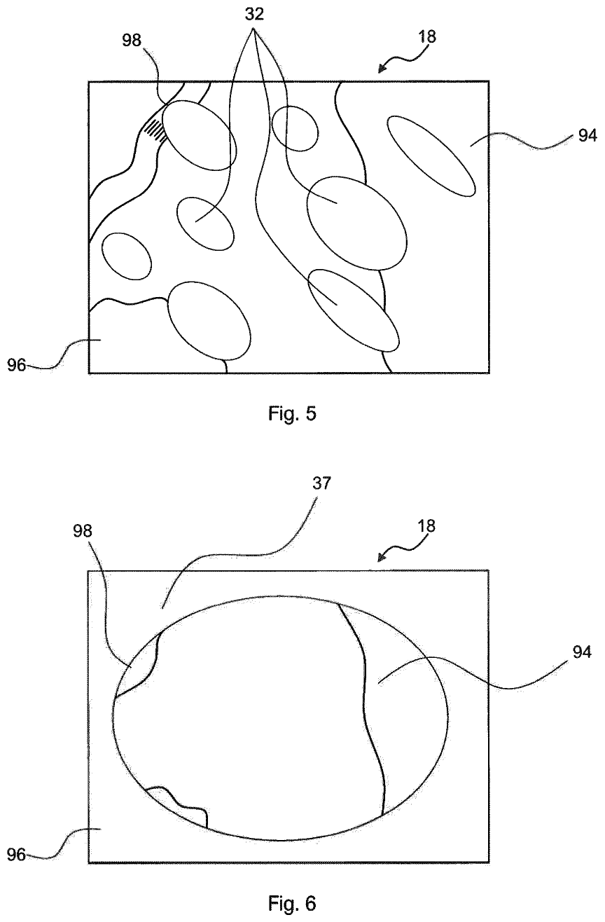

FIG. 5 is a further schematic representation of the image region to illustrate another possible fault;

FIG. 6 is a further schematic representation of the image region to illustrate another possible fault;

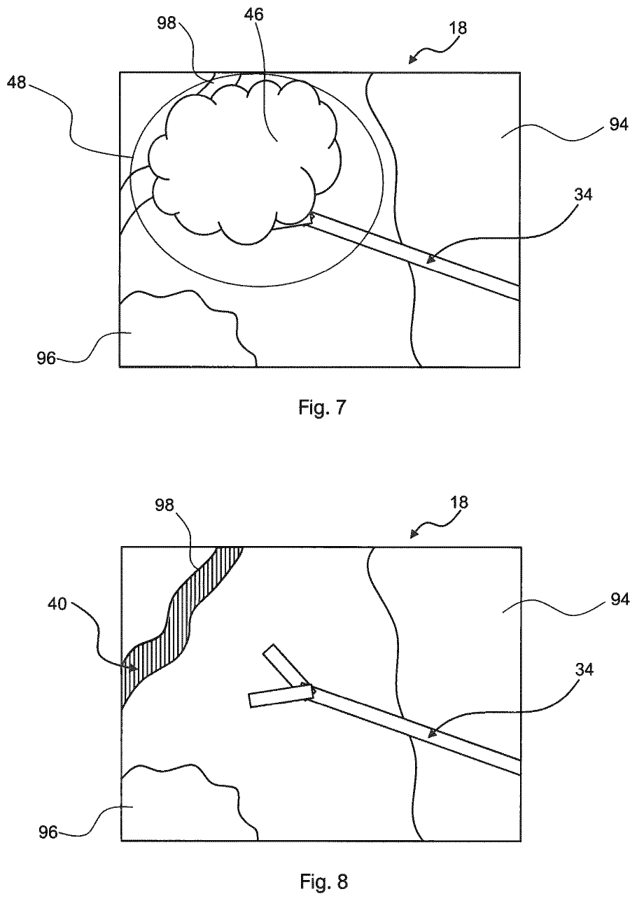

FIG. 7 is a further schematic representation of the image region to illustrate another possible fault;

FIG. 8 is a further schematic representation of the image region to illustrate another possible fault;

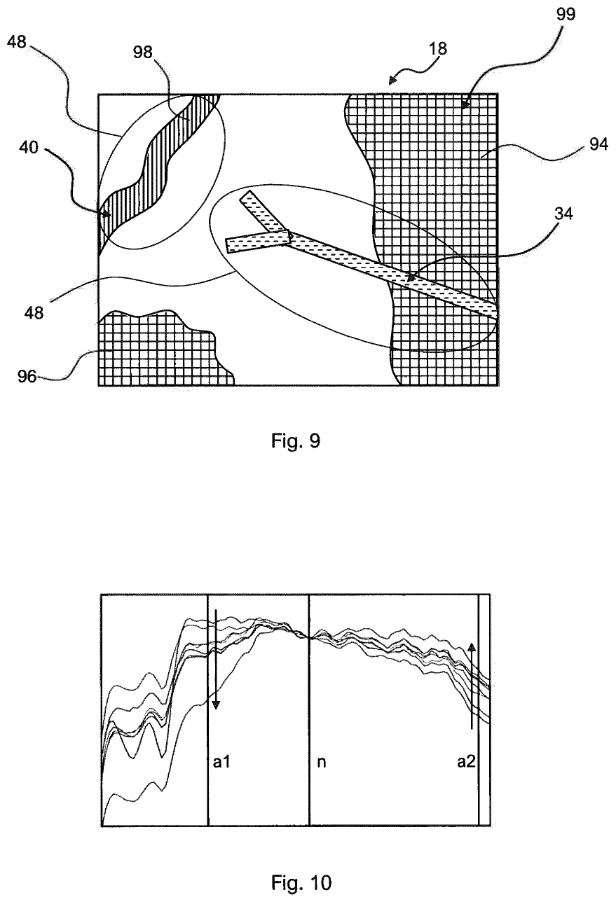

FIG. 9 is a further schematic representation of the image region to illustrate another possible fault;

FIG. 10 is a schematic representation of absorption curves of tissue with different degrees of coagulation;

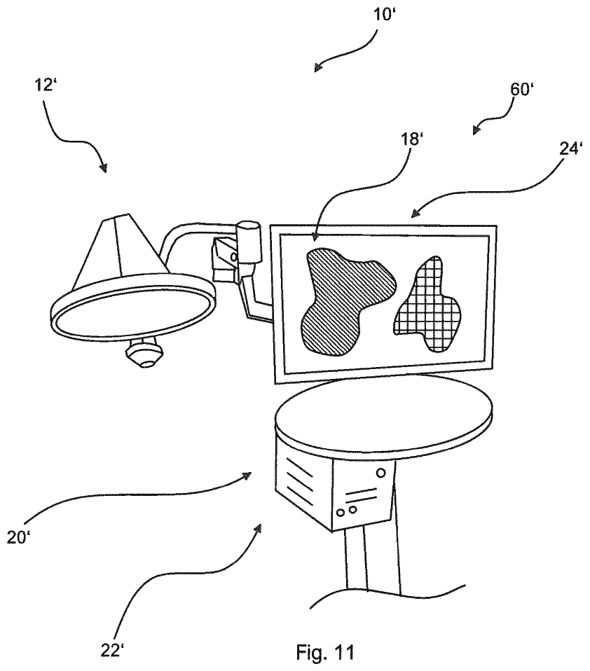

FIG. 11 is a schematic perspective view of an alternative imaging device;

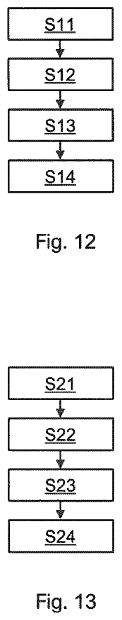

FIG. 12 is a schematic flow chart of a method for operating a medical imaging device; and

FIG. 13 is a schematic flow chart of a method for medical imaging.

FIG. 1 is a schematic representation of a medical system 60 with a medical imaging device 10. In some embodiments, the medical system 60 further includes a medical instrument 34. A schematic structural diagram of the imaging device 10 and the medical device 62 is shown in FIG. 2. Both figures are referred to in part below.

In the instance shown by way of example, the imaging device 10 is an endoscopic imaging device, specifically an endoscope device. Alternatively, the imaging device 10 could be an exoscopic, a microscopic or a macroscopic imaging device. The medical imaging device is provided for an examination of a cavity.

The medical device 34 in the illustrated case is a bipolar electrosurgical instrument. The medical instrument 34 is configured to introduce energy into tissue in a targeted manner in order to coagulate it, for example for vascular occlusion. This design is to be understood purely as an example. Other types of energy injection can be provided, as well as other types of medical devices in general, such as surgical, diagnostic, imaging, intervention-supporting, anesthetic or other medical instruments and/or devices.

The imaging device 10 comprises, for example, a medical imaging unit 64. In the shown case, this is an endoscope. Furthermore, the medical imaging device 10 may comprise a lighting device 66. In the shown case, the lighting device 66 is connected to the imaging unit 64 via a light guide. Illumination light can therefore be guided to the imaging unit 64 and directed from there onto an object to be imaged, in particular a site.

The imaging device 10 has, by way of example, a control unit 68. The control unit 68 is for example connected to the imaging unit 64 via a cable and/or an optical line and/or a light guide.

The imaging device 10 and in particular the imaging unit 64 has, by way of example, one or more windows 70 through which illumination light can be coupled out and/or object light can be coupled in.

The imaging device 64 has a distal portion 72 that includes a distal end 36. Generally speaking, this is a distal end 36 of the imaging device 10. The distal portion 72 is configured to be inserted into a cavity in an operating state. In the operating state, the distal portion 72 faces a patient in the operating state. In the operating state, the distal portion 72 is facing away from a user in the operating state. Furthermore, the medical imaging unit 64 has a proximal portion 74. In the operating state, the proximal portion 74 is arranged outside a cavity in the operating state. In the operating state, the proximal portion 74 faces away from the patient. In the operating state, the proximal portion 74 faces the user.

The imaging unit 64 has a handle 76. The handle 76 is by way of example configured for manipulation by the user. Alternatively or additionally, the handle 76 may be configured for attachment and/or connection to a medical robot. The imaging unit 64 may also be integrally formed with a robot in some embodiments. A position and/or orientation of the imaging unit 64 relative to the patient is variable, for example by manipulation by the user and/or by appropriate movement of the robot.

The medical system 60 has a display device 78. The display device 78 may be part of the imaging device 10. The display device 78 may be a separate display, such as a screen or the like. In other embodiments, the display device 78 may also be integrated into the imaging unit 64 and/or the control unit 68.

The imaging device 10 has a device interface 80 via which the medical instrument 34 may be connected to the imaging device 10. In the shown case, the device interface 80 is part of the control unit 68. In the shown case, the device interface 80 is wired. The device interface 80 is detachable. Furthermore, the device interface 80 may be configured to connect to various medical devices. The device interface 80 may comprise a socket and/or electrical connectors. In some embodiments, the device interface 80 may also be partially or completely wireless, i.e., the medical instrument 34 may then be connected wirelessly, for example via a radio connection, to the imaging device 10, and the imaging device 10 and the medical instrument 34 may have correspondingly suitable antennas.

In other embodiments, the medical instrument 34 may be entirely independent of the imaging device 10. Said imaging device may then optionally also be free of a device interface.

The imaging device 10 has a user interface 82 through which the user can make entries. The user interface 82 may comprise a plurality of controls that can be attached to different components of the imaging device 10 and/or of the medical system 60.

The imaging device 10 has a spatially and spectrally resolving image acquisition unit 12 which has at least one optical system 14. The image acquisition unit 12 further comprises an image acquisition sensor system 16 coupled to the optical system 14.

The optical system 14 and the image acquisition sensor system 16 are configured to generate image data of an image region 18. The image region 18 is shown in FIG. 1 by way of example on a display of the display device 78. The image data comprise both optical and spectral information. In the present case, the image data correspond to two-dimensional spatial data that define spatial pixels, as well as spectral data that are assigned to the individual pixels. A spectrum is therefore available for each pixel from the image data. In addition, a two-dimensional image is obtainable from the image data for each spectral band. The image data correspond to a multispectral or hyperspectral data cube.

The optical system 14 comprises optical elements (not shown) that collect object light and lead it to the image acquisition sensor system 16. The image acquisition sensor system 16 comprises a CMOS or CCD sensor (not shown). The optical system 14 and the image acquisition sensor system 16 are arranged together in a pushbroom arrangement. In other embodiments, a whiskbroom arrangement, a staring arrangement and/or a snapshot arrangement is used. In the present case, the image acquisition unit 12 is configured for hyperspectral image acquisition; the imaging device 10 is accordingly a hyperspectral imaging device. Regarding different methods of hyperspectral imaging and components required for this, reference is made to the article “Review of spectral imaging technology in biomedical engineering: achievements and challenges” by Quingli Li et al., published in Journal of Biomedical Optics 18 (10), 100901, October 2013, and to the article “Medical hyperspectral imaging: a review” by Guolan Lu and Baowei Fei, published in Journal of Biomedical Optics 19 (1), 010901, January 2014. In other embodiments, the imaging device 10 can also be multispectral. Several spectral ranges can be observed, for example, by filters that can be optionally inserted into an object light beam path and/or by sequential illumination with different wavelengths.

The image acquisition unit 12 may be at least partially included in the imaging unit 64. Parts of the optical system 14 and/or the image acquisition sensor system 16 may be included in the control unit 68. For example, object light can be guided to the image acquisition sensor system 16 via a light guide, and this can be arranged in the control unit 68. In other embodiments, the entire image acquisition sensor system 16 is included in the imaging unit 64, and only data are transmitted to the control unit 68.

The imaging device 10 further comprises an evaluation unit 20. This is configured to create an analysis of the image data. The analysis is based on both spatial as well as spectral information. The analysis includes at least one analysis parameter. This will be discussed in more detail below.

In the exemplary case, the imaging device 10 comprises an output unit 24. The output unit 24 may comprise the display device 78 and/or other output devices. In the shown case, the output unit 24 comprises, in addition to the display device 78, a loudspeaker 86 and an additional display 88 which are formed, for example, on the control unit 68. In other embodiments, the output unit 24 has an interface for connecting one or more output devices. The output generation unit 84 may also comprise the output unit 24 or its components.

The imaging device 10 comprises a control unit 90. This comprises a processor, a random access memory, a memory and appropriately configured circuits. Program code is stored on the memory and, when it is executed by the processor, causes the methods described herein to be carried out, or implements functionalities of the described units.

Furthermore, the imaging device 10 in the embodiment comprises a database 92. The evaluation unit 20 may access the database to create an evaluation of the image data. For example, the evaluation unit 20 can compare an evaluation parameter calculated by the evaluation unit 20 to parameters stored in the database 92. This will be discussed in more detail below.

The imaging device 10 also comprises a fault detection unit 22. The fault detection unit 22 is configured to detect a fault in the image acquisition and to determine a fault state of the image acquisition. The fault detection unit 22 can detect a fault state based on hyperspectral and/or multispectral information. Alternatively or additionally, the fault detection unit 22 may determine a fault state based on video image data generated by means of a video acquisition unit 28 and/or based on fluorescence imaging data generated by means of a fluorescence imaging unit 38. In some embodiments, the fault detection unit 22 may additionally or alternatively be configured to determine a fault state based on a sensor signal generated by means of a sensor unit 42. Fault detection is discussed in more detail below.

The imaging device 10 further comprises a video acquisition unit 28, which comprises a camera 30 and which is configured to generate video image data of the image region 18. The video acquisition unit 28 may also be configured to generate images in other display types, for example false color imaging, in addition to or instead of white light images. The video acquisition unit 28 acquires video image data that does not contain any spectral information. In many cases, image acquisition with spectral information takes more time than video data acquisition. Video image data can be generated and/or output in real time. Image data containing spectral information can be acquired and/or output substantially in real time or at a slower refresh rate than single images. It may be advantageous, e.g., for positioning a medical instrument 34 or for positioning the image acquisition unit 12, to operate the imaging device 10 without generating image data with spectral information. In such a case, in particular the video acquisition unit 28 can generate video image data in real time. The imaging device 10 may be multimodal in some embodiments. Image data can be generated in different modes. The different modes may be used simultaneously, alternately and/or sequentially.

In the present case, the image acquisition unit 12 is configured to generate white light images and hyperspectral images of the image region 18. White light images do not contain any spectral information. Both modes, hyperspectral imaging and white light imaging, can be performed simultaneously and/or alternately and/or sequentially. Images from both modes can be combined after imaging. Alternatively or additionally, the hyperspectral image can also be combined with video image data that is different from white light images.

The imaging device 10 also comprises a fluorescence imaging unit 38. The fluorescence imaging unit 38 is configured to acquire the image region 18 by means of fluorescence imaging and to generate fluorescence imaging data. For example, a contrast agent 40 may be used to make a specific type of tissue recognizable by fluorescence imaging. Fluorescence imaging data generation by means of the fluorescence imaging unit 38 represents a mode that may be used simultaneously, alternately and/or sequentially with other modes. The fault detection unit 22 is configured to detect a fault based on an assessment of the fluorescence imaging data. In particular, the fault detection unit 22 can detect the presence of contrast agent 40 in at least a part of the image region 18.

According to the exemplary embodiment, the imaging device 10 also comprises a sensor unit 42 which comprises at least one sensor 44. The sensor 44 is configured to measure a measured variable that describes a state of the imaging device 10 and/or an environment of the imaging device 10. The fault detection unit 22 is configured to detect the presence of a fault based on the sensor signal.

The sensor 44 may, for example, be an acceleration sensor. The acceleration sensor measures a movement of the imaging device relative to the image region 18. The fault detection unit 22 is configured to determine a range of motion based on the sensor signal and to detect the presence of a fault when the determined range of motion exceeds a threshold value.

In another exemplary embodiment, the sensor 44 may be a distance sensor. This is designed to determine a distance between the imaging device 10 and an object, which is not shown in detail and is located in the image region 18. Depending on the distance, optimized imaging can be achieved either automatically or after output via the output unit 24 by a user.

In the present application example, spatially and spectrally resolved image data are acquired using the pushbroom method. The image acquisition by means of the imaging device 10 using the fault detection unit 22 is explained with reference to FIGS. 3 to 10. Fault detection is not limited to the pushbroom method. For example, if image data are acquired point by point using the whiskbroom method, fault detection can also be applied. Furthermore, not all possible faults are listed. The faults shown in the following figures are selected merely as examples to explain the functioning of the fault detection unit 22.

FIG. 3 shows a schematic representation of an image region 18. The image region 18 is observed, for example, in the context of a microinvasive procedure. For this purpose, the distal portion 72 of the imaging unit 64 (see FIG. 1) is inserted into a cavity. The image region contains various native structures 94, 96 as well as a vessel 98, for example a blood vessel, which is to be sealed by means of a medical device 62.

First, spatially and spectrally resolved image data of the image region 18 are generated. For this purpose, the image region 18 is illuminated, and object light coming from the image region 18 is then detected. In the present case, the detected spectral information concerns light absorption. For each pixel, an absorption spectrum can correspondingly be obtained. Image data of the image region 18 are presently at least also recorded by the video acquisition unit 28. Image data of the image region 18, which are generated by means of the video acquisition unit 28, are available at a higher repetition rate than the spatially and spectrally resolved image data. The user can observe the image region 18 using the video image data during the generation of spatially and spectrally resolved image data via the output unit 24 in real time. The fault detection unit 22 has access to spatially and spectrally resolved image data as well as to video image data.

In addition, the distance of the imaging device 12 to at least one object in the image region 18 and/or the relative movement between the imaging device 12 and at least one object in the image region 18 is detected. The object mentioned may be, for example, the medical instrument 34 and/or the native structure 94, 96 and/or the vessel 98.

If, as in the present example, the vessel 98 is to be sealed by means of the medical device 62, a high quality of the hyperspectral imaging of the vessel 98 is of great importance for assessing the quality and success of the procedure. If a fault caused by a large relative movement between the imaging device 12 and the vessel 98 is detected by the sensor unit 42, the user is informed of the fault state. Said user may then be asked to perform a new hyperspectral imaging of the image region 18.

The evaluation unit 20 is configured to analyze the spatially and spectrally resolved image data. The evaluation comprises, for example, an image segmentation and a comparison of spectral information to information that is saved in the database 92 and concerns properties of various structures, tissue entities, etc. Regions detected in the context of image segmentation can be analyzed for their spectral properties. The database 92 stores, for example, which tissue entities are typically characterized by which absorption spectra and/or show certain absorption values for certain spectral bands, in particular relative to other spectral bands.

The evaluation unit 20 creates at least one analysis parameter. The analysis parameter may, for example, indicate which tissue entity is in the corresponding image segment.

During the evaluation, the medical instrument 34 can also be detected. The presence of an inorganic material 26, such as the medical instrument 34 in this example, may cause a fault in image acquisition. The medical instrument 34 can be recognized and assigned using hyperspectral information. For this purpose, known spectral properties of the inorganic material in question can be taken into account.

A low degree of error susceptibility can be achieved if video image data generated with the video acquisition unit 28 in addition to the hyperspectral image acquisition are examined for the presence of inorganic material 26 by an Al algorithm. This allows a high degree of accuracy in the detection of inorganic material 26 to be achieved.

Furthermore, on the basis of the analysis, an overlay 99 is generated, which can be displayed to the user via the output unit 24 (see FIG. 1). As illustrated in FIG. 4, the different detected tissue entities are highlighted differently, for example by overlaying a colored overlay, a texture, a label, or a combination. The user can therefore easily understand which partial regions, instruments, organs, vessels, nerves, fascia, etc. are located in the image region 18. Creating the analysis and generating the overlay can be performed repeatedly. As mentioned, video image data can form the basis for this. The overlaid highlights of different tissue structures based on spectral information can be updated substantially in real time.

The medical instrument 34 is detected by the fault detection unit 22 based on video image data and spatial and spectral information. In the present example, the medical instrument 34 can be assigned to data in the database 92 based on the spectral information and is recognized by means of a corresponding comparison. The accuracy of the assignment is increased by the video image data. If there is non-assignable inorganic material in the image region 18, it is detected by the fault detection unit 22 and, for example, marked without overlay 99 or in color, in particular black, and in any case displayed without spectral parameter representation. A faulty partial region 48 in which inorganic material is present is detected, and the faulty partial region 48 is identified via the output unit. This is done, for example, by circling the faulty partial region 48 in color.

As explained, the fault detection of inorganic material 26 according to this application example is carried out using two types of image data. On the one hand, inorganic material 26 is detected using spectral information. The fault detection therefore takes place sequentially after the image acquisition. On the other hand, inorganic material 26 is detected from video image data using an Al algorithm. The fault detection takes place before and/or during, in particular simultaneously with, the image acquisition of the spatially and spectrally resolved image data.

Another possible fault is shown in FIG. 5. The same image region 18 can be seen as in the previous figures, i.e., structures 94, 96 and the vessel 98 can be seen. In addition, one can see a fault due to soiling and/or fogging 32 of the optical system 14 (see FIG. 2) in the image region 18. The depicted soiling and/or fogging 32 is detected from video image data using an Al algorithm. According to this example, the user is prompted to clean the optical system 14 in order to achieve flawless imaging. According to other embodiments, an automatic cleaning of the optical system 14 can be performed after detection of a fault due to soiling and/or fogging 32.

FIG. 6 again shows the same image region 18 with the known structures 94, 96 and the vessel 98 to illustrate another possible fault. Only a detail of the image region 18 can be clearly seen, because, according to this example, the distal end 36 is still partially located within a trocar 37, which is used to introduce the imaging device 10. Consequently, in FIG. 6 a part of the interior of a trocar 37 can be seen in the image region 18. Based on video image data, the trocar 37 is recognized in the image region 18. The user may be prompted to correct the fault, for example by further advancing the distal end 36 through the trocar 37 for imaging.

In FIG. 7, the image region 18 with the structures 94, 96 and the vessel 98 as well as the medical instrument 34 is shown a short time, for example a few seconds, after coagulation of the vessel 98. The coagulation of the vessel 98 has produced smoke 46, which is present between the vessel 98 and the optical system 14 and can be seen in the image region 18. The smoke 46 means that spectral information of the vessel 98 obscured by the smoke 46 is not recorded and/or at best is recorded as having faults. The smoke 46 can be detected using spectral information. Alternatively or additionally, the smoke 46 can be detected using video image data. In the case of video image data, a suitable Al algorithm, such as binary classification with ResNet 50, is used for smoke detection. A combination of both detection methods increases the reliability and accuracy of the detection. Image data points associated with smoke 46 are identified to the user. In addition, the faulty partial region 48 of the image region 18 is optically highlighted on the output unit 24, for example by a circling in color. The user is also prompted to initiate the acquisition of the spatial and spectral information of the image region 18 again, optionally after observing a suitable waiting time. Alternatively or additionally, the user may be prompted to activate a smoke extraction system not shown.

In FIG. 8 and FIG. 9, the image acquisition of the image region 18 is shown with additional use of the fluorescence imaging unit 38. The vessel 98 is colored using a fluorescent contrast agent 40. The fluorescence imaging unit 38 detects the vessel 98 colored with contrast agent 40, highlighted by the hatching in FIG. 9, and displays it to the user in combination with a white light image. In addition, spatial and spectral information of the image region 18 is recorded, evaluated and displayed to the user by means of overlays 99.

The faulty partial region 48 in which contrast agent 40 is present and which is detected by the fluorescence imaging unit 38 and/or fault detection unit 22 is excluded from the evaluation of the spatial and spectral information. In this region, tissue detection may yield erroneous results or fail due to the contrast agent.

In addition to fluorescent contrast agent 40, inorganic material 26 can be seen in the image region 18. This is detected in the manner already described by the fault detection unit 22 on the basis of spectral information and video image data. This application example illustrates the multimodal nature of the invention. Spatial and spectral information is acquired simultaneously with white light images, video image data and fluorescence image data. The fault detection unit 22 can therefore detect different types of faults, in the case of FIG. 9 fluorescent contrast agent 40 and inorganic material 26, simultaneously, and the imaging device 10 can display tissue entities by means of overlays 99 in partial regions of the image region 18 which are not subject to any detected fault.

It is understood that fault detection can be carried out at any time, in particular at the same time as image acquisition.

FIG. 10 shows schematic absorption curves in the visible and near-infrared range of tissue that was coagulated with different parameters. Such curves can be obtained from spatially and spectrally resolved image data as part of an evaluation, for example by averaging over the image region 18 or a suitably selected partial region. The analysis may include absorption curves instead of a parameter image as described above or in addition to it. The analysis parameter in this case is, for example, the spectrally resolved intensity.

The inventors have found that the absorption curves for less and more strongly coagulated tissue behave differently in different spectral ranges. In a short-wave range, which is indicated by the line a1 in FIG. 10, the absorption decreases with increasing coagulation. In a long-wavelength range, which is indicated by line a2 in FIG. 10, the absorption increases with increasing coagulation. The spectra in FIG. 10 are normalized, in this case by division by the particular absorption value at line n. As described below, based thereon, an analysis and assessment can be made of whether a coagulation treatment has been completed with sufficient quality.

The analysis is based on a before-and-after comparison, wherein image data serves as a reference before the diagnostic and/or therapeutic activity is performed. The before-and-after comparison includes a difference calculation that indicates the extent to which the absorption has changed in the considered spectral ranges. To compensate for effects that are due to different amounts of tissue in the image region being observed or other deviations, the spectra to be compared are normalized as illustrated in FIG. 10. In the present case, for example, the differences between lines a1 and a2 are determined as evaluation parameters.

Which ratios, differences and/or other relationships between these evaluation parameters can provide information about the quality of coagulation are saved in the database 32. The evaluation unit 24 used this information as evaluation information and, based thereon, assesses the evaluation parameters obtained from the analysis. In the present case, in this assessment, a binary attribute is determined that can assume the values “coagulation complete” and “coagulation incomplete”.

If there is a fault in the acquisition of the spatially and spectrally resolved image data on the basis of which such absorption curves are generated, for example one of the faults described above, the absorption curves may not be usable. One or more faults can be detected by the fault detection unit 22 as described above. It is understood that the faults are not, or at least not exclusively, detected on the basis of the analysis parameter used, but independently thereof.

Subsequently, a user output can be generated based on the analysis parameter in accordance with a detected fault state. For example, an absorption spectrum is only displayed and/or marked as usable if no fault state is present.

An alternative imaging device 10′ can be seen in FIG. 11. For clarity, the reference signs of this embodiment are provided with single quotation marks. The alternative imaging device 10′ is part of a medical system 60′ that is configured to perform exoscopic imaging. For example, this medical system 60′ can be used to observe a surgical site during a surgical procedure. The medical system 60′ basically has the same functionality as the previous embodiment. The medical system 60′ comprises an image acquisition unit 12′, an evaluation unit 20′, a fault detection unit 22′, an output unit 24′, via which an image region 18′ as well as faults and overlays etc. are displayed, with regard to the functioning of which, reference is made to the above explanations.

FIG. 12 shows a schematic flow chart of a method for operating an imaging device. The sequence of the method is also clear from the above explanations.

The imaging device 10, 10′ described above is operated by way of example.

The method comprises a step S11 of acquiring spatially and spectrally resolved image data of an image region 18 by means of the medical imaging device 10, 10′.

Furthermore, the method comprises a step S12 of creating an analysis of the spatially and spectrally resolved image data that is based on spatial and spectral information and based on at least one analysis parameter calculated from the spatially and spectrally resolved image data.

Furthermore, the method comprises a step S13 of carrying out a fault detection, in which, independently of the analysis of the image data and the calculated analysis parameter, the presence of a fault in the image acquisition is detected and in which a fault state of the image acquisition is determined.

Furthermore, the method comprises a step S14 of generating a user output according to the fault state based on the analysis parameter.

FIG. 13 shows a schematic flow chart of a method for medical imaging. The sequence of the method is also clear from the above explanations.

By way of example, the method is carried out by means of the imaging device 10, 10′ described above.

The method comprises a step S21 of performing an image acquisition of an image region 18 in which spatially and spectrally resolved image data are generated which comprise both spatial and spectral information.

In addition, the method comprises a step S22 of creating an analysis of the spatially and spectrally resolved image data that is based on spatial and spectral information and on at least one analysis parameter calculated from the spatially and spectrally resolved image data.

Furthermore, the method comprises a step S23 of carrying out a fault detection, in which, independently of the analysis of the image data and the calculated analysis parameter, the presence of a fault in the image acquisition is detected and in which a fault state of the image acquisition is determined.

Furthermore, the method comprises a step S24 of generating a user output in accordance with the fault state based on the analysis parameter.

As can be seen from the above, the sequence of steps S11 to S14 or S21 to S24 is not necessarily consecutive. FIGS. 12 and 13 are to be understood as purely exemplary in this regard.

### LIST OF REFERENCE SIGNS

10 Imaging device
12 Image acquisition unit
14 Optical system
16 Image acquisition sensor system
18 Image region
20 Evaluation unit
22 Fault detection unit
24 Output unit
26 Inorganic material
28 Video acquisition unit
30 Camera
32 Soiling and/or fogging
34 Medical instrument
36 Distal end
37 Trocar
38 Fluorescence imaging unit
40 Contrast agent
42 Sensor unit
44 Sensor
46 Smoke
48 Faulty part
60 Medical system
64 Imaging device
66 Lighting device
68 Control unit
70 Window
72 Distal portion
74 Proximal portion
76 Handle
78 Display device
80 Device interface
82 User interface
84 Output generation unit
86 Loudspeaker
88 Display
90 Control unit
92 Database
94 Structure
96 Structure
98 Vessel
99 Overlay

1. A medical imaging device, in particular an endoscopic and/or exoscopic and/or microscopic imaging device, comprising:
a spatially and spectrally resolving image acquisition unit comprising at least one optical system and at least one image acquisition sensor system coupled to the optical system, which are configured to carry out an image acquisition of an image region, in which unit spatially and spectrally resolved image data are generated which comprise both spatial and spectral information;
an evaluation unit which is configured to create an analysis of the spatially and spectrally resolved image data that is based on spatial and spectral information and based on at least one analysis parameter calculated from the spatially and spectrally resolved image data;
a fault detection unit which is configured to detect the presence of a fault in the image acquisition and to determine a fault state of the image acquisition independently of the analysis of the image data and the calculated analysis parameter; and
an output unit configured to generate a user output based on the analysis parameter in accordance with the fault state.

2. The medical imaging device according to claim 1,
wherein the fault detection unit is configured to detect the presence of a fault based on an assessment of the spatially and spectrally resolved image data.

3. The medical imaging device according to claim 2,
wherein the fault detection unit is configured to detect an image exposure state based on the spatially and spectrally resolved image data and to detect the presence of a fault if the exposure state represents an underexposure and/or an overexposure at least in portions.

4. The medical imaging device according to claim 2,
wherein the fault detection unit is configured to detect the presence of a fault by detecting inorganic material in the image region.

5. The medical imaging device according to claim 1,
further comprising a video acquisition unit which comprises a camera and which is configured to generate video image data of the image region,
wherein the fault detection unit is configured to detect the presence of a fault based on an image analysis of the video image data.

6. The medical imaging device according to claim 5,
wherein the fault detection unit is configured to determine a range of motion of the camera relative to the image region based on the image analysis of the video image data and to detect the presence of a fault if the determined range of motion exceeds a threshold value.

7. The medical imaging device according to claim 5,
wherein the fault detection unit is configured to detect the presence of soiling and/or fogging on at least part of the optical system of the spatially and spectrally resolving image acquisition unit based on the image analysis of the video image data and to detect the presence of a fault in accordance with the detection of soiling and/or fogging.

8. The medical imaging device according to claim 1,
wherein the fault detection unit is configured to assess, based on a comparison of the spatially and spectrally resolved image data and the video image data, whether a white balance of the spatially and spectrally resolving image acquisition unit and a white balance of the video acquisition unit are consistent at least within a predetermined tolerance and to detect the presence of a fault if the predetermined tolerance is exceeded.

9. The medical imaging device according to claim 5,
wherein the fault detection unit is configured to detect the unexpected presence of a medical instrument in the image region based on the image analysis of the video image data and to detect the presence of a fault in accordance with the detection of an unexpected presence of a medical instrument.

10. The medical imaging device according to claim 9,
wherein the imaging device comprises a distal end, which comprises at least parts of the optical system of the image acquisition unit,
wherein the fault detection unit is configured to detect that the image region at least partially comprises an interior of a trocar, and
wherein the output unit is configured to generate a user output containing the information that the distal end is at least partially located within the trocar.

11. The medical imaging device according to claim 1,
further comprising a fluorescence imaging unit which is configured to acquire the image region by fluorescence imaging and to generate fluorescence imaging data,
wherein the fault detection unit is configured to detect the presence of a fault based on an assessment of the fluorescence imaging data.

12. The medical imaging device according to claim 11,
wherein the fault detection unit is configured to detect the presence of contrast agent in at least a portion of the image region based on the fluorescence imaging data, and
wherein the output unit is configured to generate the user output based on the analysis parameter such that parts of the image region in which the fault detection unit detects a presence of contrast agent are omitted.

13. The medical imaging device according to claim 1,
further comprising a sensor unit having at least one sensor which is configured to measure at least one measured variable which describes a state of the imaging device and/or an environment of the imaging device, and which unit is configured to generate a sensor signal which represents the measured variable,
wherein the fault detection unit is configured to detect the presence of a fault based on the sensor signal.

14. The medical imaging device according to claim 13,
wherein the sensor is an acceleration sensor, wherein the sensor signal represents a movement of the imaging device, and wherein the fault detection unit is configured to determine a range of motion based on the sensor signal and to detect the presence of a fault when the determined range of motion exceeds a threshold value.

15. The medical imaging device according to claim 13,
wherein the sensor is a distance sensor configured to determine a distance between the imaging device and an object in the image region.

16. The medical imaging device according to claim 1,
wherein the fault detection unit is configured to detect the presence of smoke in the image region based on an image analysis and to detect the presence of a fault in accordance with the detection of smoke in the image region.

17. The medical imaging device according to claim 1,
wherein the fault detection unit is configured to prevent the acquisition of spatially and spectrally resolved image data by means of the spatially and spectrally resolving image acquisition unit in the event of a fault being detected.

18. The medical imaging device according to claim 1,
wherein the output unit is configured to generate a spatially resolved parameter representation of the image region in accordance with the analysis parameter and to be identified by the fault detection unit as faulty parts of the image region.

19. The medical imaging device according to claim 1,
wherein the output unit is configured to provide the user, in the event of a fault being detected, with information on how to remedy the fault.

20. A method for operating a medical imaging device,
wherein the medical imaging device comprises a spatially and spectrally resolving image acquisition unit which comprises at least one optical system and at least one image acquisition sensor system coupled to the optical system, which are configured to carry out image acquisition of an image region in which spatially and spectrally resolved image data are generated which comprise both spatial and spectral information,
comprising the steps of:
acquiring spatially and spectrally resolved image data of an image region by means of the medical imaging device;
creating an analysis of the spatially and spectrally resolved image data that is based on spatial and spectral information and based on at least one analysis parameter calculated from the spatially and spectrally resolved image data;
performing a fault detection in which, independently of the analysis of the image data and the calculated analysis parameter, the presence of a fault in the image acquisition is detected and in which a fault state of the image acquisition is determined; and
generating a user output according to the fault state based on the analysis parameter.

21. The method for medical imaging, in particular carried out with a medical imaging device according to claim 20, comprising the steps of:
performing an image acquisition of an image region in which spatially and spectrally resolved image data are generated which comprise both spatial and spectral information;
creating an analysis of the spatially and spectrally resolved image data that is based on spatial and spectral information and based on at least one analysis parameter calculated from the spatially and spectrally resolved image data;
performing a fault detection in which, independently of the analysis of the image data and the calculated analysis parameter, the presence of a fault in the image acquisition is detected and in which a fault state of the image acquisition is determined; and
generating a user output according to the fault state based on the analysis parameter.
